(19) United States
(12) Patent Application Publication
(10) Pub. No.: US 2024/0090818 A1
(43) Pub. Date: Mar. 21, 2024

(54) HEALTH SENSING RETENTION BAND

(71) Applicant: Apple Inc., Cupertino, CA (US)

(72) Inventors: Javier Mendez, San Jose, CA (US); Samuel G. Smith, San Francisco, CA (US); Grant H. Mulliken, Los Gatos, CA (US); Trevor J. Ness, Santa Cruz, CA (US)

(21) Appl. No.: 18/463,217

(22) Filed: Sep. 7, 2023

Related U.S. Application Data
(60) Provisional application No. 63/376,281, filed on Sep. 19, 2022.

(51) Int. Cl. A61B 5/384; A61B 5/00; A61B 5/372

(52) U.S. Cl. CPC A61B 5/384; A61B 5/0075; A61B 5/372; A61B 5/6803; A61B 5/742

(57) ABSTRACT

A head-mountable device including a housing, a display positioned in the housing, a processor positioned in the housing, and a retention band connected to the housing, the retention band comprising a sensor configured to monitor a brain activity of a user.

### CROSS-REFERENCE TO RELATED APPLICATION(S)

This claims priority to U.S. Provisional Patent Application No. 63/376,281, filed 19 Sep. 2022, and entitled “Health Sensing Retention Band,” the entire disclosure of which is hereby incorporated by reference.

### FIELD

The described embodiments relate generally to retention bands for head-mountable devices. More particularly, the present embodiments relate to retention bands for head-mountable device including health sensors.

### BACKGROUND

Recent advances in portable computing have enabled head-mountable devices (HMD(s)) that provide augmented and virtual reality (AR/VR) experiences to users. The ever-increasing complexity and functionality of these head-mountable devices encourages constant improvements to the electrical components and sensors.

Sensors can be utilized on head-mountable devices for various purposes, such as detecting user information, user identity, biometric information, environmental detection, movement, location, etc. An arrangement of sensors that efficiently utilizes the limited real estate on an HMD and which is ideally positioned to detect the intended data is needed.

### SUMMARY

According to some aspects of the present disclosure, a head-mountable device can include a housing, a display positioned in the housing, a processor positioned in the housing, and a retention band connected to the housing, the retention band including a sensor to monitor a brain activity of a user.

In some examples, the retention band includes a sensor array positionable adjacent a back of a head of the user when the head-mountable device is worn by the user. The sensor array can detect brain activity of the user. The sensor can transmit a signal to the processor, the signal based on the biometric information. The processor can analyze the signal and cause the head-mountable device to perform an action in response to the analysis of the signal. The action can be at least one of providing a visual feedback, providing an audio feedback, or providing a haptic feedback.

In some examples, the retention band is removably attached to the housing. The sensor can be removably attached to the retention band. The retention band can be in electrical communication with the display. The retention band can be pivotably attached to the housing.

According to some aspects, a headband for a head-mountable device can include a first end to attach to the head-mountable device, a second end to attach to the head-mountable device, and a sensor positioned between the first end and the second end. The sensor can detect brain activity of a user, and generate a signal based on the detected brain activity.

In some examples, the sensor can be embedded in the headband. The headband can include an area that is transparent to the signal emitted by the sensor. The headband can be articulable relative to the head-mountable device. The headband can include an expandable surface area. The signal can include a display command signal.

According to some aspects, a wearable electronic device can include a display, a retention band attached to the display, the retention band having a first orientation and a second orientation, and a sensor connected to the retention band. The sensor can detect a brain activity of a user and produce a signal based on the brain activity. A processor can perform a first analysis of the signal in response to the retention band being in the first orientation, and can perform a second analysis of the signal in response to the retention band being in the second orientation.

In some examples, the wearable electronic device can perform an action in response to the signal. The wearable electronic device can include a head-mountable device. The retention band can be adjustable. The sensor can perform at least one of functional near-infrared spectroscopy or electroencephalography.

In some examples, the retention band is movable between a first position and a second position. The sensor can be oriented toward a first brain region in the first position, and a second brain region in the second position when the wearable electronic device is worn by the user. The retention band can produce at least one of visual, audio, or haptic feedback in response to the signal from the sensor. The sensor can be a first sensor. The wearable device can further include a second sensor connected to the retention band. The second sensor can collect vital signs.

### BRIEF DESCRIPTION OF THE DRAWINGS

The disclosure will be readily understood by the following detailed description in conjunction with the accompanying drawings, wherein like reference numerals designate like structural elements, and in which:

FIG. 1 shows a schematic block diagram of a head-mountable device.

FIG. 2 shows a top view of a head-mountable device.

FIG. 3 shows a cross-sectional side view of a head-mountable device.

FIG. 4A shows a rear perspective view of a retention band.

FIG. 4B shows a side view of the retention band of FIG. 4A in an articulated position.

FIG. 4C shows a side view of the retention band of FIG. 4A in an articulated position.

FIG. 5 shows a perspective exploded view of a head-mountable device.

FIG. 6 shows a side view of a retention band having sensors.

### DETAILED DESCRIPTION

Detailed reference to representative embodiments illustrated in the accompanying drawings is provided below. The following descriptions are not intended to limit the embodiments to one preferred embodiment. To the contrary, it is intended to cover alternatives, modifications, and equivalents as can be included within the spirit and scope of the described embodiments as defined by the appended claims.

The present disclosure details a retention band for a head-mountable device that is integrated with health sensors. The integrated retention band can enable both optical and electrical neural imaging of the user's head. In some examples, the sensor-integrated headband can be used for brain imaging. For example, the sensors can be configured to perform functional near-infrared spectroscopy (FNIR). In some examples, the sensors can be used to perform EEG (electroencephalography).

The retention band can be adjustable or articulable to position the sensors at desired locations on the user's head. By having the ability to adjust the retention band, the sensors can observe specific areas of the brain. For example, the position of the sensors can be tuned or tailored to look at brain areas related to language, learning, memory, comprehension, sleep, stress, pain, attention, fear, discomfort, etc.

In some examples, the retention band can be integrated with a sensor array that includes one or more transmitters that emit a signal. The transmitter(s) can be positioned a certain distance away from one or more detectors that sense the emitted signal. Based on the received signals by the detectors, a processor can infer or determine certain brain activity. In some examples, the integrated sensors on the retention band form a brain-computer interface (BCI), such as a non-invasive neural interface. In some examples, the integrated sensor array can be used to detect the parieto-frontal network of the brain.

The HMD can include an output or feedback module that provides feedback based on the detections from the sensors. The feedback from the system can include displaying visualizations to the user. For example, the feedback can include breathing visualizations, visualization that relate to the user's attention, promptings or suggestions to the user to take a break or change an activity, or make other recommendations or notifications to the user.

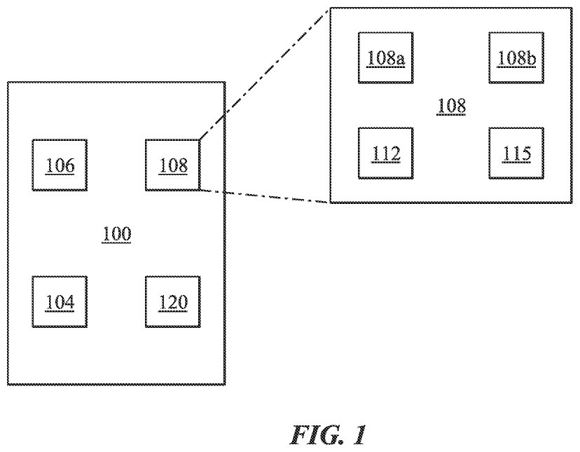

These and other embodiments are discussed below with reference to FIGS. 1-6. However, those skilled in the art will readily appreciate that the detailed description given herein with respect to these Figures is for explanatory purposes only and should not be construed as limiting. Furthermore, as used herein, a system, a method, an article, a component, a feature, or a sub-feature comprising at least one of a first option, a second option, or a third option should be understood as referring to a system, a method, an article, a component, a feature, or a sub-feature that can include one of each listed option (e.g., only one of the first option, only one of the second option, or only one of the third option), multiple of a single listed option (e.g., two or more of the first option), two options simultaneously (e.g., one of the first option and one of the second option), or combination thereof (e.g., two of the first option and one of the second option).

FIG. 1 shows a schematic block diagram of a head-mountable device 100 including a display 104, a retention band 108, a memory 106, and a processor 120. The display 104 can include one or more optical lenses or display screens in front of the eyes of a user. The display 104 can present an augmented reality visualization, a virtual reality visualization, or other suitable visualization to a user. Additionally, the display 104 can be positioned in or on a housing or frame that supports the display 104 and additional electronic components. The display 104 in combination with the frame or the housing and other electronic components supported by the housing can be referred to as the display unit 104 and the display 104. The retention band 108 can be connected to the frame or housing of the display 104.

In some examples, the retention band 108 can include a support, strap, headband, belt, arms, or any other attachment mechanism capable of supporting the HMD 100 on the user's head. In some examples, the retention band 108 can include a pliant (or semi-pliant) material or fabric that wraps partially around the user's head.

The retention band 108 can be made from a flexible material and can securely and snuggly fit around a head of the user. The retention band 108 can be made from a woven fabric, leather, polymer, or any other material compatible to be integrated with the sensors 112 or having electromagnetic transmissive properties. In some examples, the retention band 108 can be made from silicone or thermoplastic polyurethane (TPU). In some examples, the retention band 108 can be made from compression molded materials, such as rubber. The sensors 112 can be integrated into the compression molded materials. In some examples, the retention band 108 is semi-rigid. For example, the retention band 108 can be rigid where the sensors 112 are located. The sensors 112 can be flexible or deformable relative to each other such that the sensor array can move, flex, or stretch with the retention band 108. The retention band 108 can include rigid sections where electronic components are located.

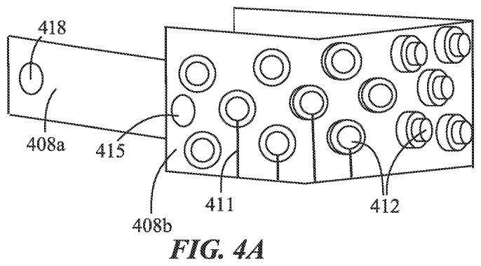

The retention band 108 can include two ends, straps or sidebands 108a that connect to the display 104. The retention band 108 can include a middle section 108b that is positioned between the ends 108a of the retention band 108. The middle section 108b can configured to contact the back of the user's head, while the sidebands 108a can be considered to contact the sides of the user's head. The retention band 108 can include pivots 115. The pivots 115 can be located between the display 104 and the sidebands 108a such that the entire retention band 108 can pivot or rotate relative to the display 104. In some examples, the pivots 115 can be positioned between the sidebands 108a and the middle section 108b to enable the middle section 108b to pivot or rotate relative to the sidebands 108b and display 104. Further detail of the pivots will be provided below with regard to FIGS. 4A-4C.

The retention band 108 can include one or more sensors 112. It will be understood that throughout the disclosure, reference to “sensors” can refer to one or more sensors. The phrase “sensors” allows for, but does not necessarily require, multiple sensors. In some examples, the sensors 112 can be removably attached to the retention band 108. The retention band 108 can be removably attached to the display 104 and in electrical communication with the display 104. In other words, the sensors 112 can be in electrical communication with the display 104.

The sensor 112 of the retention band 108 can collect biometric information, such as brain activity and vital signs (including body temperature, pulse data, respiration data, and blood pressure). The sensor 112 can generate a signal based on the collected user information and transmit the signal to the processor 120 to be analyzed. In some examples, the memory 106 can include programmed instructions responsive to the signals from the sensors 112. In some examples, the instructions can cause an output or feedback module to perform an action in response to the signal (i.e., in response to the biometric information collected by the sensor 112). In some examples, the retention band 108 itself provides feedback (e.g., through LEDs, tactile or haptic feedback, changing its shape, color, etc.) based on the collected biometric information.

For example, a user may be in a meditation or mental awareness session that is facilitated by the HMD 100. During the activity, the user's brain waves or other biometric may change in a way that is detectable by the sensors 112. The sensors 112 can generate one or more signals based on the received input. The sensors 112 can transmit the signals to the processor 120, which may cause an action by one or more components of the HMD 100 (e.g., to the display 104 to provide a visual feedback to the user). The display 104, being in electrical communication with the retention band 108, can receive electrical communication and provide feedback to the user related to their biometric readings (e.g., visual feedback, audio feedback, haptic feedback, etc.). Feedback can include determining when the user needs to take a break, or when the difficulty of an activity needs to be lowered or raised, the output can include scheduling various activities for the user or recommending adjustment of the HMD 100.

The sensors 112 can include a variety of different sensing devices. The sensors 112 can include, but are not limited to, brain activity sensors, a camera or imaging device, temperature device, oxygen device, movement device, brain activity device, sweat gland activity device, breathing activity device, muscle contraction device, etc. In some examples, the sensor can detect or sense biometric features including features of the autonomic nervous system.

Some particular examples of sensors include an electrooculography (EOG) sensor, electrocardiography (ECG or EKG) sensor, EEG (electroencephalography), photoplethysmography (PPG) sensor heart rate sensor, hear rate variability sensor, blood volume pulse sensor, oxygen saturation (SpO2) sensor, compact pressure sensor, electromyography (EMG) sensor, core-body temperature sensor, galvanic skin response (GSR) sensor, functional near-infrared spectroscopy (FNIR) sensor, functional magnetic infrared imaging (FMRI) sensor, non-contact passive infrared (IR) sensor, accelerometer, gyroscope, magnetometer, inclinometer, barometer, infrared sensor, global positioning system sensor, etc.

In some examples, the sensors 112 can include, contact microphones (e.g., pressure-based MEMS), bioelectrical activity sensors, UV exposure sensors, or particle sensors. In some examples, certain sensors can be used to assess stress and emotion. In some examples, the sensors can operate through coin cell battery or Bluetooth connectivity. In some examples, the sensors are powered by a primary battery of the HMD.

The sensors 112 described herein can allow for observations of the autonomic nervous system (ANS), to observe relaxation and stress indicators, mental health, medical treatments, etc. Using the sensors, physicians and care takers could have live feedback of biometrics. Use cases can include fitness settings, user content, workplace, telepresence, clinical, education, training, pain, therapy, etc. In some examples, the sensors can be used to capture facial expressions. This is particularly relevant given the user's face is covered by HMD. For example, the HMD could use MEMS or motion tracking sensors to detect facial expressions.

In some examples, the sensors 112 can be used to perform EEG detections. The sensors 112 can measure the electrical activity in the cerebral cortex (the outer layer of the brain). The sensors 112 can include electrodes that are placed on a participant's head, then the electrodes can non-invasively detect brainwaves from the subject. The EEG sensors 112 can record up to several thousands of snapshots of the electrical activity generated in the brain every second. The recorded brainwaves can be sent to amplifiers, then to the processor 120, a remote electronic device, or the cloud to process the data.

In some examples, the sensors 112 can be used to perform functional near-infrared spectroscopy (FNIR). The sensors 112 can use low levels of non-ionizing light to record changes in cerebral blood flow in the brain through the optical sensors 112 placed on the surface of the scalp. The signals can be recorded via flexible fiber optic cables.

The HMD 100 can be a wearable device can include electronic components that are communicatively coupled to each other and to the sensors 112 via a wired or wireless communications link. The communications link can be a physical connection, such as an electrical wire, or can be a wireless connection, such as Bluetooth, Wi-Fi, proximity sensors, etc. In some examples, the HMD 100 can be communicatively coupled to a companion electronic device, such as a remote, or a personal computing device such as a smart phone, a smart watch, a laptop, a tablet, an HMD, or any other form of electronic device. As described in further detail below, the signals from the sensors 112 can influence the HMD 100. For example, the sensors 112 can influence the visual information, content, style, frequency, and operation of the content provided by the sensors 212.

Any of the features, components, and/or parts, including the arrangements and configurations thereof shown in FIG. 1 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIG. 1.

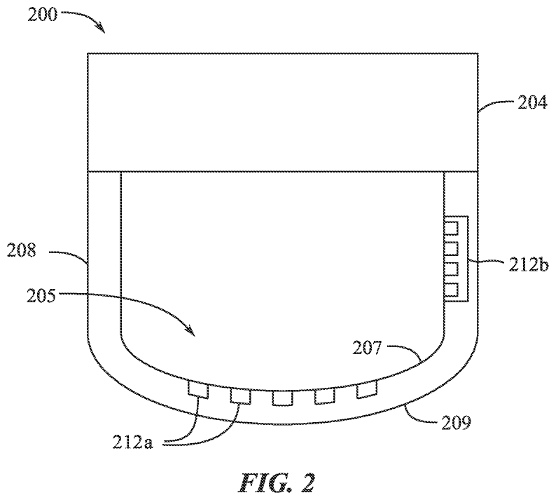

FIG. 2 shows a top view of a head-mountable device (HMD) 200. The HMD 200 can be substantially similar to, and can include some or all of the features of, the head-mountable devices described herein, such as HMD 100. The HMD 200 can include a display unit 204 and a retention band 208. The display unit 204 can include electronic components, such as a display, processor, memory, controller, battery, and other circuitry and electronics. The display unit 204 can include a frame or housing structurally supporting the electronic components. In some examples, the display 204 includes an opaque, translucent, transparent, or semi-transparent screen, including any number lenses, for presenting visual data.

The frame can at least partially border one or more edges of the display. In some examples, the frame can be configured to contact a user's head or face. In some examples, the frame blocks outside light and limits the peripheral view of the user. Various components of the display unit 204 can be housed within the frame. For example, the hardware and electronics which allow functionality of the HMD can be housed within the frame. In some examples, the frame can include attachment mounts for connecting the display unit 204 to the retention band 208. For example, the frame can include mechanical attachments, magnetic attachments, or any other suitable connector to attach the retention band 208 to the display unit 204.

The HMD 200 can be worn on the user's head such that the display unit 204 is positioned over the user's face and disposed over one or both of the user's eyes. In some examples, the retention band 208 can be positioned against (i.e., contacting or pressing against) the side and/or back of a user's head when worn. In some examples, the retention band 208 can be at least partially positioned above the user's ear or ears. In some examples, the retention band 208 can be positioned adjacent to the user's ear or ears. The display unit 204 and the retention band 208 can form a loop defining an aperture 205 for the user's head. It should be understood, however, that this configuration is just one example of how the components of an HMD 200 can be arranged, and that in some examples, a different number of connector straps and/or retention bands can be included.

In some examples, the retention band 208 includes a first sensor array 212a including one or more sensors. The sensor array 212a can be positioned on the back of the retention band 208, located to contact or be proximate the back of the user's head when the HMD 200 is donned by the user. In some examples, the retention band 208 includes a second sensor array 212b including one or more sensors. The second sensor array 212b can be positioned on a side strap or arm of the retention band 208, located to contact or be proximate the side of the user's head when the HMD 200 is donned by the user. In some examples, the first sensor array 212a and the second sensor array 212b are separate and distinct, both in form and functionality. The first sensor array 212a can include different sensors than the second sensor array 212b.

In some examples, the first sensor array 212a can translate or move along the retention band 208. For examples, the first sensor array 212a can slide from a first position on the retention band 208 (e.g., the back of the band) to a second position on the retention band 208 (e.g., the side strap). Translation of the sensor array 212a can be manually or automatically actuated. In the manner, the sensor array 212a can move to monitor different regions of the user's head.

In some examples, the first sensor array 212a and the second sensor array 212b (collectively referred to as “sensors 212”) include the same sensors and are considered part of a single array of sensors. The sensors 212 can be positioned on an interior surface 207 of the retention band 208. In some examples, the sensors 212 are positioned in the retention band 208 (i.e., between the interior surface 207 and the exterior surface 209. In some examples, the sensors 212 can span substantially across an entirety of a length of the retention band 208. As will be discussed in greater detail herein, the sensors 212 can be integrated with the retention band 208 in a variety of ways.

In some examples, the sensors 212 can be used to perform EEG detections. The sensors 212 can measure the electrical activity in the cerebral cortex (the outer layer of the brain). The sensors 212 can include electrodes that are placed on a participant's head, then the electrodes can non-invasively detect brainwaves from the subject. The EEG sensors 212 can record up to several thousands of snapshots of the electrical activity generated in the brain every second. The recorded brainwaves can be sent to amplifiers, then to the HMD computer, a remote electronic device, or the cloud to process the data.

In some examples, the sensors 212 can be used to perform functional near-infrared spectroscopy (FNIR). The sensors 212 can use low levels of non-ionizing light to record changes in cerebral blood flow in the brain through the optical sensors 212 placed on the surface of the scalp. The signals can be recorded via flexible fiber optic cables.

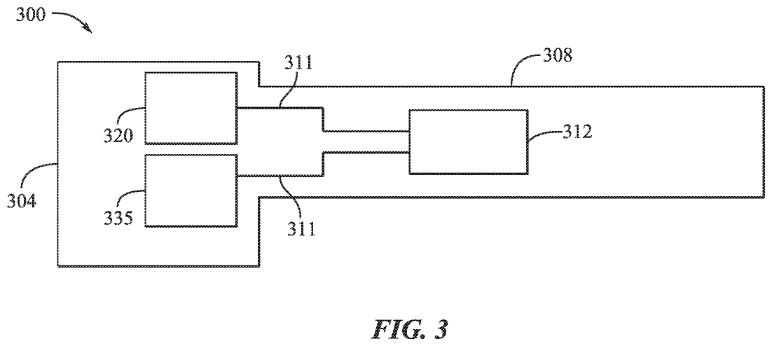

Any of the features, components, and/or parts, including the arrangements and configurations thereof shown in FIG. 2 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIG. 2. Further details regarding the integration of sensors into an HMD retention band are described below with reference to FIG. 3.

FIG. 3 shows a cross-sectional side view of a head-mountable device (HMD) 300. The HMD 300 can be substantially similar to, and can include some or all of the features of, the head-mountable devices described herein, such as HMDs 100 and 200. In some examples, sensor(s) 312 positioned in/on the retention band 308 can be operatively coupled to one or more electronic components in the display unit 304. For example, the sensors 312 can be electrically coupled a power source, such as a battery 335 that is disposed within or on the housing of the display unit 304 via an electrical connection 311. The sensor 312 can be communicatively coupled with a processor 320 or other controller that is housed within the head-mountable device 300. The battery 335 and/or the processor 320 can be located with the housing of the display unit 304, located within the retention band 108, disposed exterior to the housing of the display unit 304 and the retention band 108, or combinations thereof. The sensor 312 can generate and transmit signals reflective of the biometrics of the user to the processor 320. The processor 320 can then cause commands or instructions to be issued.

Although the sensors 312 are shown as being connected to the battery 335 and the processor 320 through a wired connection 311, in some examples, the sensor 312 can wirelessly receive data and/or power from the battery 335 and/or processor 320 by any desired method or technology. In some examples, the sensors 312 can be communicatively coupled to an external device that is not located on the HMD 300.

Further, although the components of the wearable electronic device 300 are shown as being connected to one another at certain locations, it should be understood that any of the components of the HMD 300 can be electrically and/or mechanically connected to one or more of any of the other components of the HMD 300, in any manner and location, as desired.

In some examples, the retention band 308 can include other operational or functional components in addition to the sensors 312. For example, the retention band 108 can include a battery 335 that can be in electrical communication with the display unit 304 and/or the sensors 312.

Any of the features, components, and/or parts, including the arrangements and configurations thereof shown in FIG. 3 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIG. 3.

FIG. 4A shows a rear perspective view of a retention band 408. The retention band 408 can be substantially similar to, and can include some or all of the features of, the retention bands described herein, such as retention bands 108, 208, and 308. While FIG. 4A illustrates the sensors 412 being visible on an exterior of the retention band 408, it will be understood that in some examples, the sensors 412 are concealed or hidden behind a cover or layer of the retention band 408. The sensors 412 can be configured to directly contact or touch the user's head. In some examples, the sensors 412 are non-contact sensors. In some examples, the retention band 408 is made up of multiple components. For example, the retention band 408 can include sidebands or arms 408a and a middle section or back 408b (collectively retention band 408). The back 408b can be connected at each end to the arms 408a via pivots 415.

In some examples, the sensors 412 can be interconnected (i.e., electrically connected) via wires 411. The wires 411 can further define a communications link between the display unit and the retention band 408. The sensors 412 can be disposed substantially across the entirety of the back 408b of the retention band 408. In some examples, the sensors 412 can be intentionally positioned to align with specific portions of the user's head when donned. As shown in greater detail with reference to FIGS. 4B and 4C, the retention band 408 can be articulable. By adjusting, articulating, or moving the position of the retention band 408 relative to the HMD, the sensors 412 can be positioned at various regions of the user's head.

In some examples, the sensors 412 can include a grounding sensor. For example, one or more of the sensors 412 can be a grounding sensor that is used as a reference sensor. Detections from the sensors 412 can be compared against the grounding reference sensor to analyze the detections. In some examples, the sensors 412 can included rounded edges and/or elastomeric or soft material that is transparent to the signal produced by the sensors 412. The rounded edges and soft material can help to reduce discomfort when the sensors 412 are contacting the user's head. In some examples, the geometry of the sensors 412 can be specifically designed to assist in parting the user's hair to ensure efficient contact against the user's head. In some examples, the sensors move (e.g., vibrate, rotate or oscillate) to aid in proper contact against the user's skull.

In some examples, the retention band 408 is a specialized band that can be interchanged with other headbands that are customized for different uses. For example, there can be individual custom retention bands for education, sports, industrial applications, health, learning application, training application, etc. Each different retention band can be integrated with sensors specific for its needs.

In some examples, the retention band 408 can be detachable or removable from the HMD. In other words, the HMD can include a standard headband that is replaced or modified with the retention band 408. In some examples, the retention band 408 can be an add-on component that is added to the HMD when desired or needed. The retention band 408 can include electrical/mechanical attachment mechanisms to attach to the HMD. For example, the retention band 408 can include attachment mechanisms 418 that can electrically, mechanically, and or magnetically connect the retention band 408 to an HMD. In some examples, the retention band wirelessly connects to the HMD to transmit data. The retention band 408 can include its own power source and processing capabilities.

In some examples, the retention band 408 can be expandable to reveal additional sensors 412. For example, the retention band 408 can fold or overlap itself to reduce a surface area of the retention band 408. Then when desired, the retention band 408 can be stretched, unfolded, or expanded, revealing additional sensors that were previously covered.

FIG. 4B shows a side view of the retention band 408 rotated upward. As illustrated, the back 408b of the retention band 408 can rotate relative to the side arms 408a via the pivots 415. In some examples, the entire retention band 408 can rotate relative to a housing or frame of the HMD (i.e., the pivots 415 can be located on the HMD housing). Movement of the retention band 408 can be manual or automated. In some examples, the user is prompted, guided, or instructed to move the retention band 408 into a certain position or configuration. For example, the system may prompt the user to adjust the retention band 408 to optimize the ability of the sensors 412 to detect the desired biometric data. In this configuration the sensors 412 would be positioned at or near the top of the user's head.

In some examples, the HMD include multiple retention bands 408. For example, the HMD can include a back-of-the-head band that wrap around the back of the user's head, and an over-the-head band that wraps over the top of the user's head. Each of the bands can include the sensors described herein. In some examples, the retention band 408 can be a bifurcated strap with sensors on each bifurcated section to increase the coverage area of the sensors over the head.

FIG. 4C shows a side view of the retention band 408 rotated downward. As illustrated, the back 408b of the retention band 408 can rotate relative to the side arms 408a via the pivots 415. In some examples, the entire retention band 408 can rotate relative to a housing or frame of the HMD (i.e., the side straps 408a also rotate and the pivots 415 are located on the HMD housing). Movement of the retention band 408 can be manual or automated. In some examples, the user is prompted, guided, or instructed to move the retention band 408 into a certain position or configuration. For example, the system may prompt the user to adjust the retention band 408 downward to optimize the ability of the sensors 412 to detect the desired biometric data. In this configuration the sensors 412 would be positioned at or near the bottom of the user's head, near the top of the neck. In this configuration, the sensors 412 can be used to detect information about neck muscles. In some examples, the location or position of the retention band 408 can determine what type of analysis is performed by the processor. For example, the processor can interpret a signal differently based on the detected position of the retention band 408 relative to the HMD and/or user when the signal was acquired. In some examples, the signal analysis changes based on the region of the brain being monitored by the sensors 412. In some examples, operation of the sensors themselves changes based on the position of the retention band 408. For example, different sensors can be used when monitoring different areas of the brain, or different sensor emission/detections can be used based on the location of the sensors relative to the user. According to one example, a position detecting IMU, sensor, encoder, or the like can be positioned in the retention band 408 to detect the relative position of the band and generate a signal indicative of the position. The processor can then use that signal to determine the type of analysis to be performed on the data collected from the sensors 412.

Any of the features, components, and/or parts, including the arrangements and configurations thereof shown in FIGS. 4A-4C can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIGS. 4A-4C.

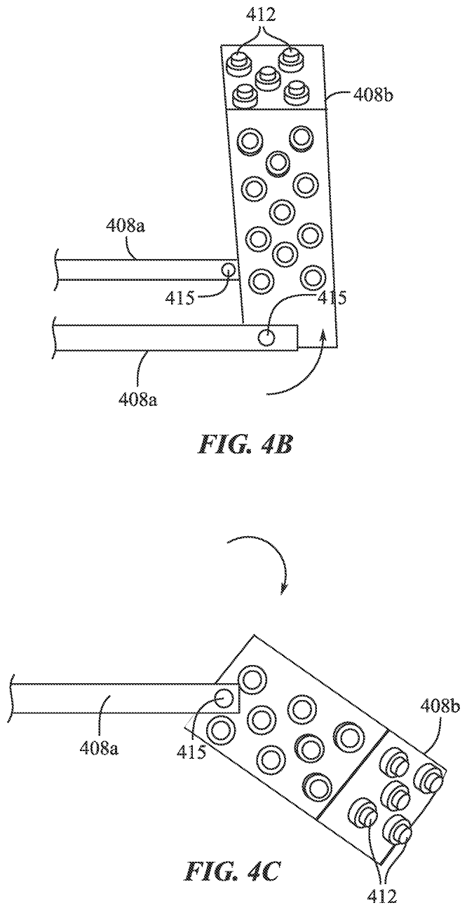

FIG. 5 illustrates a perspective exploded view of an HMD 500. The HMD 500 can be substantially similar to, and can include some or all of the features of, any of the HMDs described herein, such as HMD 100, 200, 300, and 500. The HMD 500 can include a display unit 504 and a retention band 508. The retention band 508 can include an inner portion 507 that contacts a user's head when worn, and an outer portion 509 at least partially defining an exterior surface of the wearable device 500. The outer portion 509 can be integrally formed with the inner portion 507. In other words, the inner portion 507 and the outer portion 509 can be made from a single piece of material (shown separated here for simplicity). In some examples, the outer portion 509 and the inner portion 507 can be coupled together, for example using adhesive, thread, or other coupling techniques.

In some examples, the HMD 500 can include a first sensor array 512a and a second sensor array 512b (collectively referred to as “sensors 512”). In some examples, the inner portion 507 can include a material that is transmissive to the sensors 512. The sensors 512 can be positioned between the inner portion 507 and the outer portion 509, such that the sensors 512 are positioned within the retention band 508. In some examples, the inner portion 507 of the retention band 508 can include a perforated section adjacent the sensors 512. The perforated section can include holes, gaps, or apertures formed in the material of the inner portion 507.

The holes in the inner portion 507 can correspond to locations of the sensors 512 and can allow signals from the sensors, or the sensors themselves to pass through the holes. In other words, the sensors 512 can be exposed and accessible at the inner portion 507, for example, to contact the user's head. In some examples, the sensors 512 are embedded or sandwiched between the inner portion 507 and the outer portion 509 of the retention band 508. In some examples, the sensors 512 can be position adjacent a sensorially transparent or transmissive window 530 that allows for the transmission and reception of signals between the user's head and the sensors 512. The sensors 512 can be electrically coupled to an electronic component 520 in the display unit 504, such as a processor or battery, via one or more electrical connections 511.

In some examples, the sensor arrays 512 can flexible and capable of bending or curving to according to a shape of the retention band 508 on the user's head. In some examples, the sensors 512 include flex cables and/or optical fibers or fiber optic cables that are woven into the fabric of the retention band 508. In some examples, the retention band 508 can include conductive fibers woven into the fabric of the retention band 508.

The retention band 508 can be made from a flexible material and can securely and snuggly fit around a head of the user. The retention band 508 can be made from a woven fabric, leather, polymer, or any other material compatible with micro-perforations or having electromagnetic transmissive properties. In some examples, the retention band 508 can be made from silicone or thermoplastic polyurethane (TPU). In some examples, the retention band 508 can be made from compression molded materials, such as rubber. The sensors 512 can be integrated into the compression molded materials. In some examples, the retention band 508 is semi-rigid. For example, the retention band 508 can be rigid where the sensors 512 are located.

Any of the features, components, and/or parts, including the arrangements and configurations thereof shown in FIG. 5 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIG. 5.

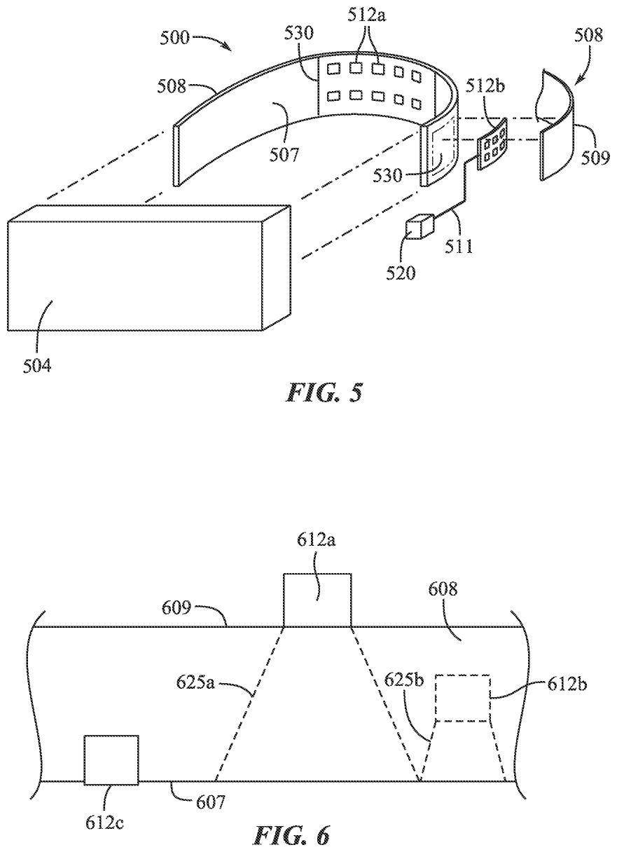

FIG. 6 shows a side or cross-sectional view of portion of a retention band 608. The retention band 608 can be substantially similar to, and can include some or all of the features of, the retention bands described herein, such as retention band 108, 208, 308, 408, and 508. The retention band 608 can include one or more sensors 612a, 612b, and 612c (collectively referred to as “sensors 612”). The sensors 612 can be partially or entirely embedded or encapsulated in the retention band 608. The retention band 608 can include a first, interior surface 607 facing the user's head when donned, and a second, exterior surface 609 facing away from the user and defining an exterior of the retention band 608. In some examples, the first surface 607 directly contacts or touches the user's head when the HMD is donned or being worn by the user.

The sensor 612a can be positioned on the exterior surface 609 of the retention band 608. In some examples, the first surface 607 can define an interior and the second surface 609 can be an exterior of the retention band 608. Thus, the sensor 612a can be positioned on an exterior of the retention band 608. In some examples, the retention band 608 can include a sensor 612b that is embedded, encapsulated, or otherwise surrounded by the retention band 608. The retention band 608 can include, a sensor 612c that is positioned such that a portion of the sensor 612c is exposed through the inner surface 607. In other words, the sensor 612c can at least partially define an interior of the retention band 608 and can directly contact or touch the user's head. In some examples, the sensor 612c can be partially surrounded or embedded in the retention band 608. In some examples, the sensor 612c is externally attached to the retention band 608, positioned on the interior of the retention band 608.

The sensors 612a and 612b can have corresponding sensor areas 625a, 625b, respectively. The sensor areas 625a, 625b can represent a field of view or cone of influence that is transparent to a signal emitted by the sensors 612a, 612b. In some examples, the field of view of the sensors 612 can be approximately 50 degrees. The sensors 612a, 612b can detect physiological, biological, and/or biometric changes of the user's body through corresponding sensor areas 625a, 625b. For example, the sensors 612a, 612b can detect changes to a user through the material of the retention band 608. Other sensors may be added or substituted, as may be desired. In some examples, the sensor areas 625a, 625b can include a different material than the rest of the retention band 608. For example, the sensor areas 625a, 625b can be transparent to certain signals from the sensors 612a, 612b or from the user, while the remainder of the retention band 608 is not transparent or transmissive to such signals.

In some instances, the closer positioning of sensor 612b to the first surface 607 than the sensor 612a can correspondingly reduce the field of view 625b. In some examples, the sensor 612c may be flush with the inner surface 607 and/or directly contacting a user's head. This is only one example of sensor depth variation, as a plurality of sensors can be disposed on the first surface 607 of the retention band 608, or between the first surface 607 and the second surface 609 of the retention band 608.

Any of the features, components, and/or parts, including the arrangements and configurations thereof shown in FIG. 6 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIG. 6.

To the extent the present exemplary systems and methods use personally identifiable information, such use of personally identifiable information should follow privacy policies and practices that are generally recognized as meeting or exceeding industry or governmental requirements for maintaining the privacy of users. In particular, personally identifiable information data should be managed and handled so as to minimize risks of unintentional or unauthorized access or use, and the nature of authorized use should be clearly indicated to users.

The foregoing description used specific, though exemplary, nomenclature to provide a thorough understanding of the described examples. The specific details are not required in order to practice the described examples, but are described herein and presented for purposes of illustration and description only. They are not intended to be exhaustive or to limit the embodiments to the precise forms disclosed. Many modifications and variations are possible in view of the above teachings.

What is claimed is:

1. A head-mountable device, comprising:
a housing;
a display positioned in the housing;
a processor positioned in the housing; and
a retention band connected to the housing, the retention band comprising a sensor configured to detect a brain activity.

2. The head-mountable device of claim 1, wherein:
the sensor further comprises a sensor array configured to detect brain activity, the sensor array positionable adjacent a back of a head.

3. The head-mountable device of claim 1, wherein:
the sensor transmits a signal to the processor, the signal based on the detected brain activity;
the processor analyzes the signal; and
the processor causes the head-mountable device to perform an action in response to the analysis of the signal.

4. The head-mountable device of claim 3, wherein the action comprises at least one of providing a visual feedback, providing an audio feedback, or providing a haptic feedback.

5. The head-mountable device of claim 1, wherein the retention band is removably attached to the housing.

6. The head-mountable device of claim 1, wherein the sensor is removably attached to the retention band.

7. The head-mountable device of claim 1, wherein the retention band is in electrical communication with the display.

8. The head-mountable device of claim 1, wherein the retention band is pivotably attached to the housing.

9. A headband for a head-mountable device, comprising:
a first end configured to attach to the head-mountable device;
a second end configured to attach to the head-mountable device; and
a sensor positioned between the first end and the second end, the sensor configured to:
detect brain activity of a user; and
generate a signal based on the detected brain activity.

10. The headband of claim 9, wherein:
the sensor is embedded in the headband; and
the headband comprises an area that is transparent to the signal emitted by the sensor.

11. The headband of claim 9, wherein the headband is articulable relative to the head-mountable device.

12. The headband of claim 9, wherein the headband comprises an expandable surface area.

13. The headband of claim 9, wherein, the signal comprises a display command signal.

14. A wearable electronic device comprising:
a display;
a retention band attached to the display, the retention band having a first orientation and a second orientation;
a sensor connected to the retention band, the sensor configured to detect a brain activity of a user and produce a signal based on the brain activity; and
a processor configured to:
perform a first analysis of the signal in response to the retention band being in the first orientation; and
perform a second analysis of the signal in response to the retention band being in the second orientation.

15. The wearable electronic device of claim 14, wherein the wearable electronic device performs an action in response to the signal.

16. The wearable electronic device of claim 14, wherein:
the wearable electronic device comprises a head-mountable device; and
the retention band is adjustable.

17. The wearable electronic device of claim 14, wherein the sensor is configured to perform at least one of functional near-infrared spectroscopy or electroencephalography.

18. The wearable electronic device of claim 14, wherein the retention band is movable between the first orientation and the second orientation, the sensor directed toward a first brain region in the first orientation, and a second brain region in the second orientation when the wearable electronic device is worn by the user.

19. The wearable electronic device of claim 14, wherein the retention band produces at least one of visual, audio, or haptic feedback in response to the signal from the sensor.

20. The wearable electronic device of claim 14, wherein:
the sensor is a first sensor; and
further comprising a second sensor connected to the retention band, the second sensor configured to collect vital signs.
